(19) United States
(12) Patent Application Publication
(10) Pub. No.: US 2025/0191486 A1
(43) Pub. Date: Jun. 12, 2025

(54) METHOD AND APPARATUS FOR GENERATING AND PRESENTING A SKIN CARE ROUTINE

(71) Applicant: Solomomo Corporation, Redmond, WA (US)

(72) Inventors: Michael Hyman, Yarrow Point, WA (US); Zhenya Hyman, Yarrow Point, WA (US); Cindy Engstrom, Redmond, WA (US)

(21) Appl. No.: 18/537,043

(22) Filed: Dec. 12, 2023

(51) Int. Cl. G09B 5/06; A61B 5/00; G06T 7/00; G06V 40/10; G11B 27/10; G16H 30/40; G16H 40/67

(52) U.S. Cl. CPC G09B 5/065; A61B 5/40; A61B 5/741; G06T 7/0012; G06V 40/10; G16H 30/40; G16H 40/67; G06T 2207/30088; G06V 2201/03; G11B 27/10

(57) ABSTRACT

Briefly, embodiments of a system, method, and article for receiving selfie images of a patient and additional images of one or more portions of the patient's body from a skin measurement/monitoring device. The selfie images and the additional images may be processed to identify one or more skin care conditions for the patient. A group of skin care products may be determined to recommend to the patient based on the one or more determined skin care conditions. A multi-day, multi-day portion schedule for a skin care routine may be generated for the patient, the skin care routine comprising one or more products from the group of skin care products and one or more additional actions for the patient to perform. Instructions may be generated for the patient, the instructions indicating how to use the one or more products and how to perform the one or more additional actions in a particular order. The instructions may be transformed into a personalized track comprising audio and/or video, and a link to the personalized track may be provided to the patient.

### BACKGROUND

Many people/patients engage in various techniques/routines or apply different skin care products in order to maintain a healthy and/or youthful appearance. For example, a particular skin care routine may include applying various moisturizers or other products in a specific order, to specific areas of a patient's skin at certain times of the day. Such a routine may improve the health of the patient's skin and may provide benefits over time if the routine is followed and/or is adjusted periodically. However, it may be difficult, cumbersome, and expensive for a patient to maintain a skin care routine day after day, particularly if the patient lives a busy life and occasionally loses motivation to follow a prescribed skin care routine. Moreover, if the skin care routine involves a relatively large number of steps, which they typically do, or the application of a relatively large number of products, which is typically six to fifteen products, the patient may eventually lose interest in sticking to the skin care routine, thereby relinquishing the pursuit of desired outcomes.

A person may set up appointments with skin care providers such as licensed aestheticians to visit a spa, such as a medical spa, periodically over the year, to check on the status of the person's skin health and appearance. From these interactions, the care provider will often suggest a plan to treat the patient's skin issues and goals going forward into the future. The patient may set up follow up appointments at varying periodicity to return to the spa to check on the status of the skin healthcare treatments and/or to determine how well the skin care routine is working for the patient and whether the skin care routine is addressing any issues which have previously been identified for the patient. However, because human skin constantly grows and renews on average every twenty-eight days and reacts to fluid environmental and physiological conditions, if the patient's skin issues change between visits, a skin care aesthetician might not be aware of the changes for months until the next time that the patient returns to the spa.

### BRIEF DESCRIPTION OF THE DRAWINGS

Features and advantages of the example embodiments, and the manner in which the same are accomplished, will become more readily apparent with reference to the following detailed description taken in conjunction with the accompanying drawings.

FIG. 1 illustrates a system for skin care maintenance according to an embodiment.

FIG. 2 illustrates a user mobile device which may be utilized by a patient in accordance with an embodiment.

FIG. 3 illustrates a skin monitoring/measurement device which may be utilized by a patient in accordance with an embodiment.

FIG. 4 illustrates a computing device according to an embodiment.

FIG. 5 illustrates a functional block diagram of a system for determining a patient's skin care conditions according to an embodiment.

FIG. 6 illustrates a process for determining products to use for a patient's skin care routine according to an embodiment.

FIGS. 7A and 7B illustrate a process for determining a schedule for a patient's skin care routine according to an embodiment.

FIG. 8 illustrates a process for determining instructions for a particular action to be performed according to an embodiment.

FIG. 9 illustrates a system for monitoring a patient's compliance with a skin care routine and for providing rewards to the patient according to an embodiment.

FIGS. 10A and 10B illustrate a process for providing a link to a personalized audio track and/or video to a skin care patient according to an embodiment.

FIG. 11 illustrates a system for implementing a process for providing a link to a personalized audio track to a skin care patient according to an embodiment.

Throughout the drawings and the detailed description, unless otherwise described, the same drawing reference numerals will be understood to refer to the same elements, features, and structures. The relative size and depiction of these elements may be exaggerated or adjusted for clarity, illustration, and/or convenience.

### DETAILED DESCRIPTION

In the following description, specific details are set forth in order to provide a thorough understanding of the various example embodiments. It should be appreciated that various modifications to the embodiments will be readily apparent to those skilled in the art, and the generic principles defined herein may be applied to other embodiments and applications without departing from the spirit and scope of the disclosure. Moreover, in the following description, numerous details are set forth for the purpose of explanation. However, one of ordinary skill in the art should understand that embodiments may be practiced without the use of these specific details. In other instances, well-known structures and processes are not shown or described in order not to obscure the description with unnecessary detail. Thus, the present disclosure is not intended to be limited to the embodiments shown but is to be accorded the widest scope consistent with the principles and features disclosed herein.

Maintaining a healthy and consistent skin care routine is challenging for many patients, particularly for those patients new to the process and who want to improve the health and appearance of their skin but are unsure of how to do so. For example, patients may encounter many issues, such as not knowing the state of their skin, such as how healthy their skin is. Patients may also be unaware of what steps they could undertake to improve the health status of their skin and/or to keep the health status of their skin from degrading over time. Patients might also be unaware of which products to use which may be beneficial to people with the same or similar skin characteristics and/or skin issues. Patients may additionally be unaware of in which order skin care products should be applied, how often they should be applied, as well as how to apply such products. Patients may be unaware of what products are being used by other people, such as commonly utilized skin care products. Patients may want to know whether their routines should change before or after various skin treatments or travel. Patients may want to know how to change routines as their bodies age or due to normal hormone fluctuations.

In accordance with one or more embodiments, a process is provided which determines potential issues with a patient's skin health, presents skin care product suggestions to the patient, builds a daily interactive skin care schedule for the patient, and generates a personalized ritual audio track and/or video track to play to the patient. The personalized ritual audio track and/or video track may include music as well as verbalized instructions as to how the patient should apply one or more skin care products. In accordance with an embodiment, an application program may perform one or more operations of such a process. The application program, or “application,” may run on a smart phone, such as an iPhone™ or Android™ phone, and/or may be presented on a laptop or desktop computer, or some other device on which a user interface may be displayed or otherwise presented to a patient.

Information regarding a patient's potential skin care issues may be acquired from several sources, such as selfie images captured by the patient's mobile device or some other device which has camera functionality. Additional images or other measurements may be acquired from other hardware devices, such as a handheld tool which a patient may hold near the patient's face or some other portion of the patient's skin. For example, a patient may hold a handheld skin measurement device and images may be captured by the handheld skin measurement device and/or other measurements may be determined by the handheld skin measurement device, such as measurements relating to moisture levels, measurements of oiliness or dryness, and/or other indicia of skin damage or skin healthiness. Images captured by the patient, such as selfie images, or images captured by a handheld measurement device may be processed by one or more analysis modules to identify potential skin health-related conditions based on information detected in the images, such as skin tags, discolorations on the patient's skin, pore issues, patches of redness or dryness, or other areas of irritation, to name just a few examples among many. Various analysis modules may utilize one or more machine-learning techniques to identify health-related conditions of the patient's skin. The outputs from the analysis modules may be provided to one or more normalization modules which may serve to generate one or more scores for the various skin health conditions. The outputs from the one or more normalization modules may be provided to a skin condition mapper which may generate or otherwise utilize a mesh map of the patient's face, such as determined from the images captured by the patient and may indicate areas of concern on the mesh map, such as areas of irritation, dryness, redness, and so forth, to name just a few examples among many.

A patient may also fill out a survey or questionnaire, providing answers to questions about the patient's skin or any known conditions or issues the patient has with his or her skin. The patient's answers to the questionnaire may be provided to a scoring system module which may determine scores for various conditions and/or characteristics.

The outputs of the scoring system module and the skin condition mapper may be provided to a skin condition module, which may identify one or more conditions of the patient's skin. For example, the identified conditions may be presented to the patient so that the patient is made aware of any issues affecting the health of the patient's skin and how to address these issues.

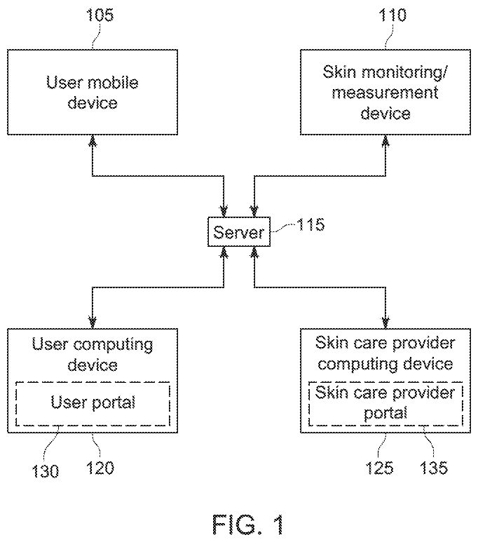

FIG. 1 illustrates a system 100 for skin care maintenance according to an embodiment. System 100 may include various devices or components, such as a user mobile device 105, a skin monitoring/measurement device 110, a server 115, a user computing device 120, and a skin care provider computing device 125. Although only five devices or components are shown in FIG. 1, it should be appreciated that more (or fewer) devices or components may be included or otherwise utilized in accordance with an embodiment. Moreover, more than one server 115 may be utilized in some embodiments.

User mobile device 105 may comprise a smart phone or some other type of portable electronic device having a camera and/or video capability. For example, a patient may utilize user mobile device 105 to take selfie images, such as of the patient's face or some other portion of the patient's skin or scalp. A “selfie image,” as used herein, refers to an image, such as a digital image, which includes oneself, either taken alone or within one or more other persons, and is taken by oneself using a device having a camera. User mobile device 105 may transmit or otherwise provide one or more selfie images to server 115. For example, a patient may periodically take selfie images of the patient's face or some other portion of the patient's skin of interest and one or more of the selfie images may be uploaded or otherwise provided to server 115. In accordance with an embodiment, a patient may utilize an application program running on the user mobile device 105 to selectively upload or transmit one or more selfie images to server 115. For example, the one or more selfie images may either be automatically uploaded or transmitted to server 115 or may be selectively uploaded, such as in response to a patient providing an input or instruction to upload or transmit one or more select selfie images to server 115.

Skin monitoring/measurement device 110 may comprise a tool which a user may utilize to acquire images of specific portions of the patient's skin and/or to take certain measurements of portions of the patient's skin or scalp. For example, skin monitoring/measurement device 110 may comprise a handheld tool which a patient may place or traverse over a portion of the patient's skin or scalp to take measurements of certain characteristics of the patient's skin, such as a moisture level or oiliness level, and that is tightly integrated with the process so that the location of images taken are known and optimized for obtaining a broad set of measurement areas, whether to get overall measurements of the face or scalp, or to repeat measurements on specific areas of concern. Skin monitoring/measurement device 110 may also include a camera for taking or acquiring images of the user's skin. For example, although user mobile device 105 may be utilized by the user to take selfie images, such images are not sufficient for detailed analysis. The skin monitoring/measurement device 110 may take images under controlled lighting conditions with multiple spectra, including infrared and ultraviolet, with controlled distances, at magnification levels that can reveal skin or scalp health details that are not available from a phone or computer camera or under ambient light. After capturing one or more images and/or taking one or more measurements, the images and/or measurements may be transmitted, uploaded, or otherwise provided to server 115. Skin monitoring/measurement device 110 may also include Wi-Fi and/or Bluetooth support with a camera and a grouping of light emitting diodes (LEDs) with different spectra including those outside the visible range and controllers. Skin monitoring/measurement device 110 may include a cone or other structure designed intentionally to keep the patient's face a certain distance from an embedded camera and may serve to block out external light to thereby acquire photos with more detail than may be possible with a smart phone taking a selfie image. For example, if the patient were to use a cell phone to take a photo of the patient while wearing red or being positioned near a red curtain, the patient's skin tones will be altered, an effect which may be reduced or otherwise eliminated via the use of skin monitoring/measurement device 110. Similarly, a photo with a cell phone may not be able to resolve hair follicles on the scalp or pore clogging on skin.

Server 115 may store images and or other measurements received from user mobile device 105 and skin monitoring/measurement device 110. Server 115 may also perform an analysis or may otherwise process the received information to make certain determinations about the condition of the patient's skin and/or determine a skin care maintenance program, as discussed in further detail below. Such an analysis performed by server 115 may be accessible to the patient and/or to a skin care provider, such as via a portal. For example, a user interface may be utilized to access the portal. The portal may comprise a website through which an analysis of the patient's skin care condition may be accessed and through which the patient may access a suggested skin care routine, including products to apply to the patient's skin or hair, an order in which to apply the products, the use of supplements or other products, the use of treatment devices or other devices, and a schedule on which the products are to be applied. The patient may also access historical photos, such as historical selfie images previously provided by the user mobile device 105 and/or images or measurements provided by skin monitoring/measurement device 110.

The patient may access a user portal 130 via a user computing device 120, such as a laptop or desktop computer. The patient may also access user portal 130 via user mobile device 105 in some implementations.

A skin care provider, such as an aesthetician, may access information about the patient's skin care via a skin care provider portal 135. For example, the skin care provider portal 135 may be accessed via skin care provider computing device 125, which may comprise a laptop or desktop computer, and/or a mobile device such as s smart phone. A skin care provider may view and observe the patient's progress over time as the patient engages in a daily skin care routine and may interact regularly with the patient, such as without the patient having to manually visit a health spa or healthcare facility for a checkup/examination.

The uploading of selfie images, and images from a skin monitoring/measuring device to server 115 may be compliant with Health Insurance Portability and Accountability Act of 1996 (HIPAA) regulations, for example. The uploading and storage of information in this manner, as well as the providing of a related analysis to both a patient and a skin care provider may provide a way of enabling the skin care provider to be able to track a patient's progress and whether the patient is following a routine prescribed by the skin care provider. Such a system may assist in avoiding situations in which a patient either forgets to use a particular product or uses the product incorrectly or should alter the products or application schedule based on results.

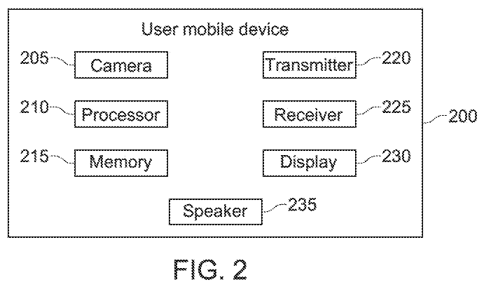

FIG. 2 illustrates a user mobile device 200 which may be utilized by a patient in accordance with an embodiment. User mobile device 200 may include various components such as a camera 205, processor 210, memory 215, transmitter 220, receiver 225, display 230, and speaker 235, to name just a few components among many possible components. A patient may utilize camera 205 to take or otherwise acquire a selfie image. Processor 210 may execute code to perform various operations and/or may execute or run one or more application programs, for example. Memory 215 may be utilized to store executable program code and/or selfie images captured by camera 205. Transmitter 220 may transmit signals or messages to devices, including transmitting the one or more captured selfie images. Receiver 225 may receive signals or messages from various devices, such as from a server. Display 230 may be utilized to present certain information to a patient, such as captured selfie images and/or other graphical information, for example. Speaker 235 may be utilized to convert electrical energy into acoustical signal energy, e.g., to present audio sound to a patient.

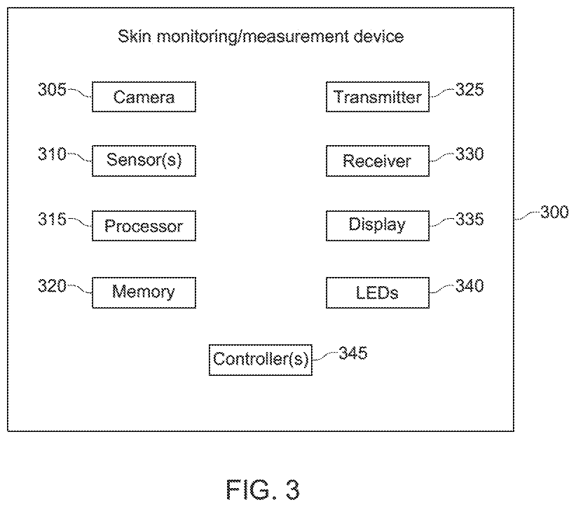

FIG. 3 illustrates a skin monitoring/measurement device 300 which may be utilized by a patient in accordance with an embodiment. Skin monitoring/measurement device 300 may include various components such as a camera 305, sensor(s) 310, processor 315, memory 320, transmitter 325, receiver 330, display 335, LEDs 340, and controller(s) 345, to name just a few components among many possible components. A patient may position skin monitoring/measurement device 300 so that it is in contact with or in close proximity to an area of skin of concern to the patient and camera 305 may be utilized to capture one or more images of the area of the patient's skin. Skin monitoring/measurement device 300 may also include sensor(s) 310 to determine various measurements of characteristics of the patient's skin in certain areas, such as measurement of dryness, oiliness, redness, and/or skin damage, to name just a few examples among many. Processor 315 may execute code to perform various operations, for example. Memory 320 may be utilized to store executable program code and/or images captured by camera 305 or measurements acquired by sensor(s) 310. Transmitter 325 may transmit signals or messages to devices, including transmitting the one or more captured images and/or measurements. Receiver 330 may receive signals or messages from various devices, such as from a server. Display 335 may be utilized to present certain information to a patient, such as one or more measurements captured by sensor(s) 310. LEDs 340 may emit or otherwise generate a certain amount of light to enable camera 305 to acquire or capture quality images of a patient's skin, and may be placed in positions to create shadowless illumination or to generate shadows from different angles for depth calculations. The skin monitoring/measurement device 300 may include additional optical elements such as diffusers or polarizers. In some embodiments, light emitting elements other than LEDs 340 may be utilized. Controller(s) 345 may perform various functions, such as controlling LEDs 340 and/or adjusting the settings on one or more other components.

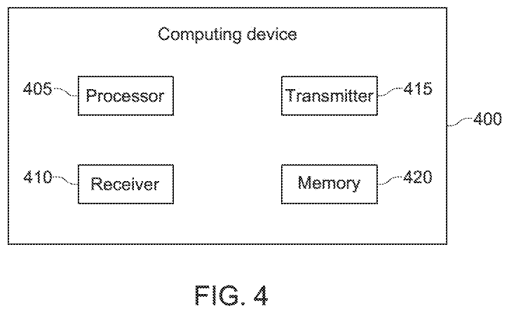

FIG. 4 illustrates a computing device 400 according to an embodiment. Computing device 400 may include various components such as a processor 405, receiver 410, transmitter 415, and memory 420. Computing device 400 may comprise a server and/or a laptop computer or desktop computer, to name just a few examples among many. Receiver 410 may receive signals or messages, such as messages comprising images and/or measurements. Receiver 410 may receive information relating to a user portal or a skin care provider portal in accordance with some embodiments. Transmitter 415 may transmit signals or messages to one or more computing devices. Memory 420 may store executable program code and/or other information such as images and/or measurements received from various devices. Processor 405 may execute code, such as code stored in memory 420 and may perform various analysis functions or operations on information relating to a patient's skin, such as images or measurements, as discussed in more detail below. In some embodiments, computing device 400 may include or may be in communication with a display and/or some other type of input/output device, such as a speaker and/or a microphone, for example.

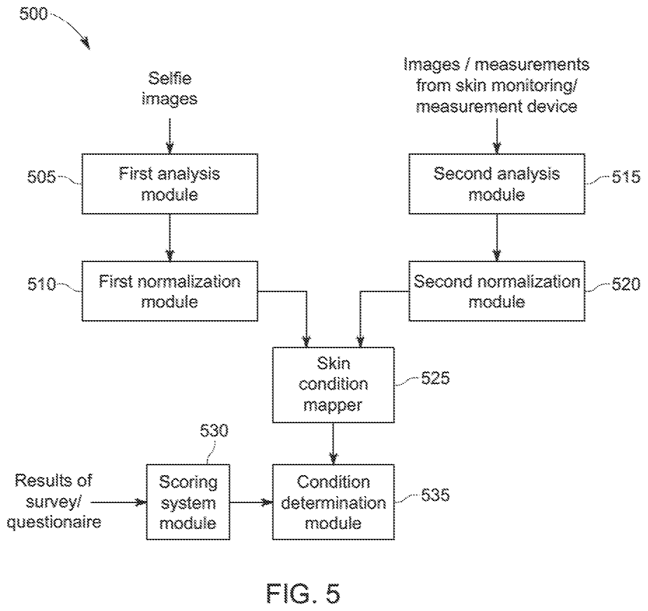

FIG. 5 illustrates a functional block diagram of a system 500 for determining a patient's skin care conditions according to an embodiment. In one particular implementation, one or more of the functional blocks shown in system 500 may be implemented by one or more computing devices including one or more servers, such as server 115 shown in FIG. 1.

In system 500, selfie images may be received by or otherwise obtained by a first analysis module 505. First analysis module 505 may implement one or more algorithms or may otherwise use machine learning processes or functionalities to determine one or more areas of concern in a patient's skin as detected from the selfie images. First analysis module 505 may determine additional information, such as areas of potential skin damage, levels of oiliness, areas of dryness or moisture, to name just a few examples among many. First analysis module 505 may determine a set of factors and scores, the factors including redness, moisture levels, pore condition, to name just a few examples, among many. Scores for each factor may also be calculated to determine how much each factor is an issue for a patient based on the selfie images. Information such as source selfie images, an algorithm vendor of an algorithm used in the analysis, an algorithm name, and an algorithm score may be determined and stored. For example, for a moisture factor, a moisture score of 0-100 may be converted to or otherwise associated with different moisture-related conditions, such as dry skin or normal skin. Accordingly, an initial set of N measurements may be converted into M different weighted conditions.

First analysis module 505 may provide results of an analysis of selfie images to first normalization module 510. First normalization module 510 may, for example, normalize or otherwise adjust scores determined for selfie images so that there is a similar value range for different algorithms employed for analysis of the selfie images. For example, raw scores determined by different algorithms may be subjected to a normalization process so that a similar value range may be determined for the processing of selfie images by different algorithms. In accordance with an embodiment, normalized scores may be determined which fall between “1” and “0,” with “1” being considered to be the best or top score and “0” to be the worst or lowest score.

Images and/or measurements from a skin monitoring/measurement device may be received by or otherwise obtained by a second analysis module 515. Second analysis module 515 may implement one or more algorithms or may otherwise use machine learning processes to determine one or more areas of concern in a user's skin detected from the images and/or measurements received from the skin monitoring/measurement device. Second analysis module 515 may determine additional information, such as areas of potential skin damage, levels of oiliness, areas of dryness or moisture, to name just a few examples among many. Second analysis module 515 may determine a set of factors and scores by employing different algorithms in a similar manner to that discussed above with respect to first analysis module, for example. Images obtained from the skin monitoring/measurement device may comprise close up direct-to-skin images. These direct to-skin images may be processed via the implementation of various algorithms to compute scores of skin condition factors in a manner similar to that for selfie images, and may be mapped to various canonical conditions and normalized to the same scale as is used from the selfie images. A “canonical condition,” as used herein, refers to condition determined from a fixed ontology. For example, the average length, width and density of wrinkles may be measured by a device measurement using a mix of machine learning algorithms. These raw scores may then be mapped to a canonical condition such as “fine lines and wrinkles” and be given a score for how well the measurements map to this particular condition, versus a canonical condition such as “deep wrinkles.”

Second normalization module 520 may, for example, normalize or otherwise adjust scores determined for images and/or measurements obtained from the skin monitoring/measurement device so that there is a similar value range for different algorithms employed for analysis of the images and/or measurements, such as to determine normalized scores between “1” and “0” for various skin condition factors.

First normalization module 510 and second normalization module 520 may output results to skin condition mapper 525. Skin condition mapper 525 may convert normalized scores received from first normalization module 510 and second normalization module 520 to an internal set of skin condition factors that are canonical across all measurements. It should be appreciated that the various modules and/or the skin condition mapper of system 500 may be implemented by a processor executing program code, for example, as part of an application program. A “module,” as used herein, refers to an extension to a main program, such as an application program, where the module is dedicated to one or more specific functions.

The first normalization module 510 and the second normalization module 520 may provide results to skin condition mapper which include a normalized compound score, such as 0.85 for a moisture factor, but may also have scores broken out by specific regions of the face or measurement area. Skin condition mapper 525 may determine separate scores for different facial regions for a particular factor, such as a moisture factor. For example, a machine learning process may be employed to compute a 3-dimensional facial mesh having a number of points, such as 300+ points, for a face and the patient's face (and corresponding processed images) may be mapped to this mesh. For example, each point on the facial mesh may correspond to or may otherwise represent a different location on the patient's face. Each point on the facial mesh may be associated with a fixed position on the patient's detected face, for example.

From the facial mesh, different mesh sections may be associated with different facial regions, such as cheeks, under eyes, forehead, or chin, to name just a few examples among many. A contributed weight of each area to an overall total for the patient's entire face may be determined in order to compute a score for each region. A user interface may direct the patient to take images from particular regions of the face, using the mesh as visual guidance.

A patient and/or a skin care provider may fill out a questionnaire for the patient, where various questions are asked which may be utilized to assist in determining a skin care maintenance or care program or plan for the patient. For example, various surveys may be completed which correspond to a number of skin type analyses. Two commonly used surveys include surveys relating to the Baumann system and the Fitzpatrick scale. The Baumann System or Baumann Skin Type Classification System refers to a skin type classification system used by skin care professionals such as dermatologists or aestheticians to divide patients into 16 distinct skin types. These 16 distinct skin types may be used to prescribe the best skin care regimen for their skin. The Fitzpatrick scale refers to a numerical classification schema for human skin color. The Fitzpatrick scale estimates the response of different types of skin to ultraviolet (UV) light. For example, the Fitzpatrick scale is a recognized tool for dermatological research into human skin pigmentation.

Other surveys may also, or alternatively, be considered in accordance with an embodiment. Each of these surveys may map a patient to a skin type, e.g. Baumann Oily, Sensitive, Non-Pigmented, and Tight (“OSNT”) or Fitzpatrick 12. For example, a score system module may determine scores for various potential skin issues based on the results of one or more surveys received for the user.

Results from the surveys or questionnaires may be provided to a scoring system module 530 which may process the results to map the answers to different skin types or other classifications. Results from scoring system module 530 and skin condition mapper 525 may be provided to a condition determination module 535. For example, conditional determination module 535 may combine scores from scoring system module 530 and skin condition mapper 525 and may convert these scores into a series of recommended product categories for a patient to address various skin condition factors which have been identified for the patient. Examples of categories include toner, moisturizer, facial sunscreen, and chemical exfoliants, to name just a few categories among many. In order to determine categories, a vector of weights may be determined for each canonical condition to map the canonical condition to the product categories. A similar table of weights may be utilized for each different skin type. For example, because certain skin types may have contraindications for certain product categories, a multiplication matrix may be employed to boost or reduce any category factor for a particular skin type. An overall factor may be utilized to indicate how to weight any skin type versus other skin types and how to weight the skin type factors versus the canonical condition factors. This approach enables a wide variety of signals from different conceptual spaces (skin types, skin proclivities, detailed skin image analysis) to be combined into a single vector of weights for different products or treatments.

If the patient has had computations determined for skin care conditions in the past, then there may be a history of which products the patient has been using for a period of time, such as for at least one month, then those computations may be considered when determining a current product recommendation. For example, calculations from the previous predetermined number of days, such as the previous 28 days may be utilized. 28 days may be considered because, for example, it takes the average adult skin cell 28 days from formation deep within dermal layers to make its way to the skin's surface before dying and shedding. It should be appreciated that if there are multiple measurements near a data point, those measurements may be averaged for that data point, for example. The previous calculations may be compared against current measurements. For example, if there is determined to be a canonical condition which has dropped by at least a threshold percentage or amount, that condition may be boosted by a factor of K (where K=. 5 will move to the midway point between the previous and last measurement average) so that treatment of that skin care condition may be continued. It should be appreciated that a value of K may vary by condition so that for certain conditions that are transient (such as acne) or which may be considered positive (e.g., clear skin) K may be 0.

For example, Relation 1 as shown below may be utilized to calculate a score for a particular canonical skin condition. If a patient has, for example, redness on her cheeks and eyebags, a skin care routine may be generated which instructs the user to apply moisturizer and eye cream. Relation 1 may be calculated periodically, such as every 28 days, to determine how the patient's skin is healing and/or changing in response to the patient following a prescribed skin care routine. Because a given measurement on a given day may be an outlier, when scores are computed for conditions, it may be desirable to reduce measurement variations through a smoothing process that takes into account past and recent measurements. In Relation 1, computation of each of the conditions scores is shown, where Ci represents a particular condition value in the full vector of conditions, and a new value is computed for a given time range by weighting previous values. Here, t represents the time of the sequence, Ki is a combining coefficient used for the particular condition Ci to control how much the previous signals are weighted versus a current signal. The averaged trailing n values is also considered, where n specifies a time window range, versus those of the previous 28 day cycle. This drives a smoothing curve that can work on a small set of data so that changes of a condition may be viewed while minimizing outlier signals and giving priority to recency, although there are other curve fitting algorithms which may also be employed to achieve a similar result, such as ARISA or cubic spline models where a weighted average is computed between a projected value and an actual value, once an extensive measurement set has been acquired for a particular patient.

$${Cnew}_{i} ( t = 0 ) = {C}_{i} ( t = 0 ) + {K}_{i} * max ( 0 , \frac{1}{2 n} {\sum }_{j = - 28 - n}^{- 28 + n} {C}_{i} ( t = j ) - \frac{1}{n} {\sum }_{j = - n}^{0} {C}_{i} ( t = j ) ) \qquad [ Relation 1 ]$$

The next time that measurements are taken and Relation 1 is calculated, scores may be different. For example, if the patient's eyebags are lower and wrinkles are higher on the patient's face, a different and/or modified skin care routine may be determined. For example, a determination may be made as to whether to keep the patient on the same routine as was used during the previous 28 days because it has worked, or whether the skin care routine should be modified to focus more on addressing wrinkles because a wrinkle factor now has a higher score as determined via implementation of Relation 1.

Multiple vectors for different canonical conditions may be combined to determine weights for different product categories. The weights for different product categories may be sorted and the top M products may be selected, where M is based on a user preference for the overall number of different products. As part of this product selection process, if a particular category has a much higher weight than the next category, a product for the higher weighted category may be doubled, depending on the category type. In one implementation, 4 or fewer products may be selected for men and six or fewer products may be selected for women, although the total number of products per person may vary per patient.

Relation 2, as shown below, illustrates an equation for determining the products for a particular patient to used based on vector scores for the patient. Relation 2 is a matrix which maps a condition weight, skin type weight, or other factor to a set of weights for particular categories, and the previously described weight computation for conditions and types is employed to compute a vector of scores for each category.

$$\begin{matrix} & Condition i & Type j \\ Category 1 & Weight 1 & Factor 1 \\ Category 2 & Weight 2 & Factor 2 \\ \cdot & \cdot & \cdot \\ \cdot & \cdot & \cdot \\ \cdot & \cdot & \cdot \\ Category N & Weight N & Factor N\end{matrix}$$

$$↓$$

$${\sum }_{n = 1}^{N} {\vec{C}}_{i} \cdot {\vec{W}}_{i} \times {\sum }_{k = 1}^{K} {\vec{T}}_{k} \cdot {Tw}_{k} \qquad [ Relation 2 ]$$

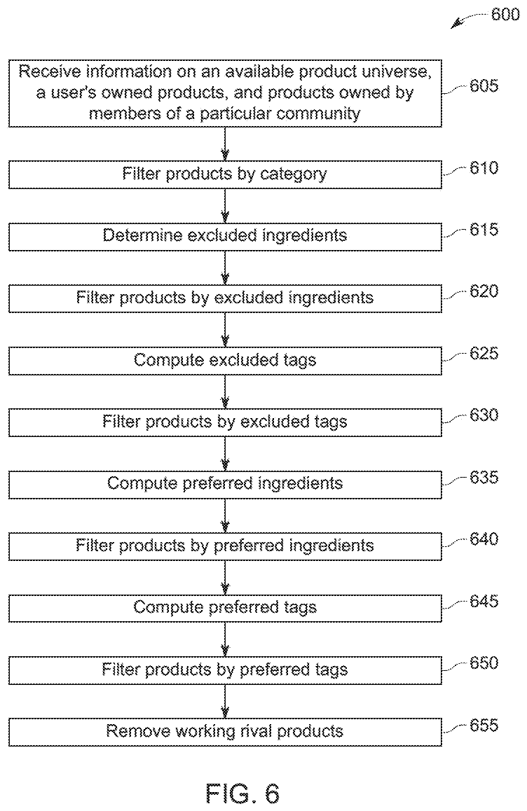

FIG. 6 illustrates a process 600 for determining products to use for a patient's skin care routine according to an embodiment. Embodiments in accordance with claimed subject matter may include all of, less than, or more than blocks 605 through 655. Also, the order of blocks 605 through 655 is merely an example order. For example, a method in accordance with embodiment 600 may be performed by a computing device having one or more processors.

At operation 605, information may be received regarding an available product universe, the products currently owned by the patient, and/or products owned by members of a particular skin care community. For example, the available product universe may comprise a group of all known skin care products which are available for purchase. The patient's currently owned products may be determined by the patient notifying a system of the skin care products currently owned by the patient. For example, the patient may manually enter the names of the patient's currently owned products by filling out a form or questionnaire. In some embodiments, the patient may capture photos of the products owned by the patient, such as by taking photos of a shelf of products owned by the patient, or the patient taking photos of a bathroom sink area where the patient stores skin care products and the skin care products may be automatically determined from the photos such as, for example, via a machine learning process.

If there is a sufficiently large and/or robust community of skin care treatment members for whom product usage data and/or preferences is known or is capable of being determined, information relating to products used by the community of skin treatment members may be utilized, in part, to determine which products to recommend for use by a particular skin care patient. For example, one or more similarity algorithms may be utilized to determine which products are used by skin care treatment members who have skin types and/or skin conditions similar to those of the particular skin care patient.

During the consideration of a product set selection, if there are a sufficiently large number of community ratings for skin care products (e.g., determined using a rating feature), better rated products may be given higher scores in the product selection algorithm. Unlike a classic similarity algorithm (e.g., cosine similarities), here the process may combine an expert rule system along with user ratings and patterns. For example, a consideration set of products may be reduced based on a domain knowledge rule system, such as where the domain rule system may indicate that for a particular skin type, a patient should not use cleanser but should instead use moisturizing water and/or ceramides. The similarities algorithm may be applied or implemented as part of a weighting process. Doing the order of these operations in reverse may remove too many products from the consideration set and lead to either no or lower value choices for certain categories.

At operation 610, products may be filtered by category. For example, products may be organized based on categories such as moisturizers, exfoliators, soaps, and eye and/or face masks, to name just a few examples among many.

At operation 615, excluded ingredients may be determined. For example, if the patient has previously submitted a list of ingredients to which the patient is known to be allergic, the identities of these ingredients may be determined from the previously submitted information. In some embodiments, one or more skin care professionals may have a list of ingredients which they recommend patients avoid using. Moreover, if certain ingredients are subject to recall notifications, the identities of these ingredients may also be determined and added to a list of excluded ingredients.

At operation 620, the products may be filtered based on excluded ingredients. For example, the system may determine which products contain the excluded ingredients and exclude those products from further consideration.

Each product in a product catalog may have metadata including usage instructions, ingredients, and keyword descriptors of products (tags). For example, a cleanser might be a foaming cleanser (and thus tagged with “foaming”). These tags may be determined from a vendor's catalogs or by a team of subject matter experts and may be maintained as part of the product catalog. A supplement might have a category (e.g., Vitamin K3-D2), a form (capsule) and a set of tags (GMO free, dye free). In operation 625, excluded tags may be determined. For example, a patient might not want any GMO supplements or may not want foaming cleansers.

At operation 630, products may be filtered based on the excluded tags. For example, products associated with excluded tags may be excluded from further consideration.

At operation 635, preferred ingredients may be determined. For example, if the patient has previously submitted a list of ingredients which the patient likes or prefers, the identities of these ingredients may be determined from the previously submitted information. In some embodiments, one or more skin care professionals may have a list of ingredients that they recommend patients use. For example, a professional may recommend coconut oil or vitamin A as ingredients for certain skin conditions.

At operation 640, the products may be filtered based on preferred ingredients. For example, the system may determine which products contain the preferred ingredients and may give preferential treatment to those products over products which do not include the preferred ingredients.

At operation 645, preferred tags may be determined. Just as a patient may have tags they want to exclude, the patient may also have tags that he or she prefers. These preferred tags can be determined explicitly through user configuration or implicitly from answers to surveys or from skin types. At operation 650, products may be filtered based on the preferred tags. For example, products associated with preferred tags may be given preferential treatment over products which are not associated with the preferred tags.

At operation 655, working rival products may be removed from consideration. For example, there may be some products which work in a similar way, such as two different brands of moisturizers. If, for example, the patient already owns one of the brands of moisturizer, the other brands of moisturizers may be removed from consideration.

Brand preferences may also be filtered in some implementations. For example, these may be set per patient or from a service vendor, such as a spa, associated with the patient. For example, if a patient is known to like La Mer™ products, and goes to Spa 1 (which carries ZO™ products) and also works with an Amway™ representative (who promotes Amway™ products), this information may be used to filter for La Mer™, ZO™ and Amway™ products.

If historical data is available, previously selected products may be preferred for continuity, although this may be changed based upon the measured results. This consideration set of potential products for a given category may thereafter be stored and used to select products for a patient for a time range. For example, products may be selected from the consideration set at random or based on other selection criteria.

In accordance with an embodiment, product selections for a patient's skin care routine may be based at least partially on patient and/or skin care provider preferences. Product selections may be altered over time based on measured changes in the patient's skin condition and/or based on changing patient and/or provider preferences.

Product and/or treatment recommendations may be based on products and/or treatments used by influencers and experts within a skin care community, for example. A full set of weights, products, and boost/reduce factors may be stored in “personality” banks, for example. A default set of values may be used for products and/or treatments, but different values may be stored for different influencers, for example. In accordance with an embodiment, a weighting arrangement may be generated for a particular aesthetician (e.g., aesthetician 1) and for a particular influencer (e.g., influencer 1). If a patient wants to get a set of recommendations for products and/or treatments, the patient may acquire a default system set, or the patient may request recommendations from a community member. If the patient requests a product or skin care treatment recommendation from a community member, the community member's weight set (e.g., for aesthetician 1 or for influencer 1) may be used to feed an algorithm. Accordingly, the use of the community member's weight set may indicate what the community member would suggest for the patient given the patient's skin measurements and the member's value set, all without any direct interaction from the community member to the patient.

After products have been determined for a patient to use, other actions may be determined for the patient. For example, there may be certain steps which would be desirable for the patient to perform during and/or after using certain skin care products. In accordance with an embodiment, it may be desirable for the patient to clean the patient's face and use a skin monitoring/measuring device to take detailed skin images, for example. There may be certain steps desirable for the patient to perform before using a skin care product and certain steps desirable for the patient to perform after using the skin care product. There may additionally be certain steps which may be desirable for the patient to perform between using different skin care products or while using a particular skin care product. To assist the patient, a skin care routine may be determined, such as via an automated process, for example. The determined skin care routine may indicate various steps for the patient to perform as part of the routine. In accordance with an embodiment, there may be different steps for a patient to perform at different times of the day, such as during the morning, during the afternoon, and during the evening. A multi-day schedule may be determined for the patient, such as a weekly and/or monthly schedule. In some implementations, a schedule may be determined which includes the same steps at the same time of day for the patient. In certain implementations, a schedule may be determined which includes different steps and/or a different order of steps on different days of a multiday schedule.

For example, a calendar matrix may be created for a patient for a multi-day period. The calendar matrix may include an order of actions or things that a patient should do as part of a treatment plan as well as a frequency of how often the various actions should be performed. These actions can include use of skin care products, hair care products, dietary supplements, medical products, or hydration or exercise routines, for example.

In one particular embodiment, preliminary steps may be generated, such as informing the patient to drink water to hydrate or to follow a certain diet. Next, there may be a moisturizer step which involves applying moisturizer in combination with another action, such as shining a blue or red light on the patient's skin via a light therapy device which may be beneficial to the patient prior to applying moisture, for example. The patient may also be directed to wash his or her face with a particular soap or cleaner prior to applying the moisturizer in some instances.

The patient may utilize an application program, such as an application which is run on the patient's smart phone, such as an iPhone™ or Android™ smart phone, or through a desktop application which provides a user interface. The schedule may be accessible through the application and the user may be prompted to provide certain inputs to indicate that the patient has performed steps of the schedule so that the patient's progress may be tracked over time.

The patient may be instructed to take a selfie photo, then wash the patient's face, and then take a detailed skin image with a skin monitoring/measurement device such as described above with respect to FIG. 3. The selfie images, skin images, and an indication that the patient washed the face may be uploaded to a portal and stored as part of the patient's records.

An example of other steps may be for the patient to brush the patient's face with a particular type of brush such as a Clarisonic™ or pore cleansing brush and then apply a cleaner and take a selfie or skin monitoring/measurement device photo. There may be additional steps, such as instructing the patient to take certain vitamins or perform certain exercises, as well.

After a multi-day schedule has been created for a patient, the schedule may be modified periodically based on the patient's progress in addressing any particular issues the patient may have. For example, if the patient has acne or a rash which is improving based on a particular scheduled routine, steps may be modified because they might not be needed as the patient's skin condition improves. In one aspect, a skin care provider, such as an aesthetician or dermatologist, may modify or replace any of the steps, products or treatment devices used and/or reorder one or more steps of the patient's scheduled routine.

The steps may be presented to a user via a user interface. For example, the steps may be presented as text in the form of a written instruction. In some implementations, audible instructions may be presented. In accordance with a particular embodiment, a text-to-audio conversion may be performed to generate an audio version of the text. A generative artificial intelligence (AI) feature or service may be utilized to help generate instructions. In some implementations, a patient may select a desired language from available languages and may select whether an audible voice is that of a man or a woman.

In some implementations, instructions may be presented in the form of a video, such as a video showing the patient how to perform a particular step of a routine. For example, a generative AI process may generate an animation depicting how a particular step is to be performed or may accompany the generative audio track with a generative video based on the patient's aesthetician or other provider.

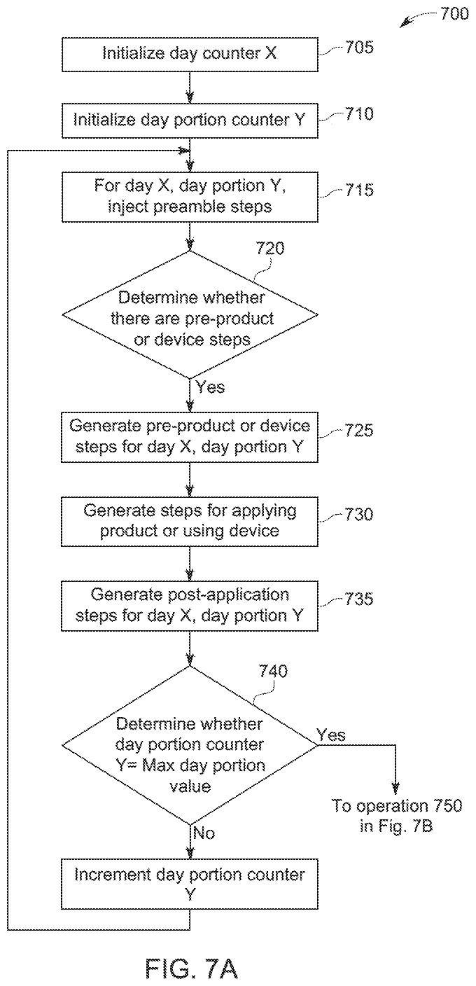

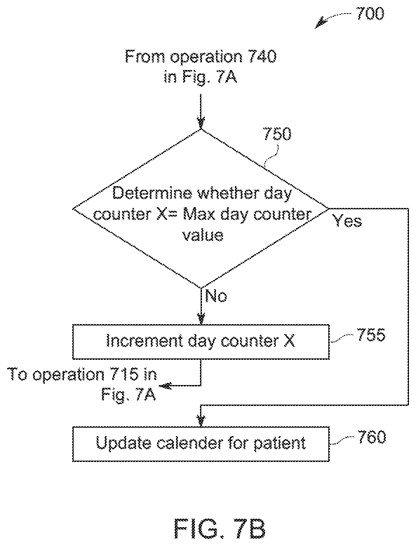

FIGS. 7A and 7B illustrate a process 700 for determining a schedule for a patient's skin care routine according to an embodiment. Embodiments in accordance with claimed subject matter may include all of, less than, or more than blocks 705 through 760. Also, the order of blocks 705 through 760 is merely an example order. For example, a method in accordance with embodiment 700 may be performed by a computing device having one or more processors.

Process 700 may be utilized to determine a multi-day schedule including multiple routines per day, such as a routine for the morning, a routine for the evening, and/or a routine for the afternoon, for example. Process 700 as described below may be used to determine a multi-day routine where each day has the same number of different routines at different times of the day for a patient, such as two routines per day, where one routine is for the morning and a second routine is for the evening. Equally, process 700 could determine a multi-day schedule having different numbers of routines for one or more days relative to the number of routines determined for a different day. In one implementation, one day of a multi-day schedule may include two routines at different times of the day, whereas a different day of the multi-day schedule may include three or more routines, for example.

At operation 705, a Day Counter X may be initialized. At operation 710, Day Portion Counter Y may be initialized. For example, Day Counter X and Day Portion Counter Y may each be initialized to an integer value of 1. A value of Day Counter X of 1 may indicate the first day of a multi-day schedule. A value of Day Portion Counter Y of 1 may indicate the first day portion of a multi-routine schedule for a particular day of the multi-day schedule. For example, the first day portion of a particular day may comprise the morning of the day.

At operation 715, for Day X, Day Portion Y (as indicated by respective Day Counter X and Day Portion Counter Y), preamble steps may be injected. “Preamble steps,” as used herein, refers to steps to be performed prior to the full product and treatment regimen. These could include preparatory washing and image taking, or could include preparing products before using them.

At operation 720, a determination is made as to whether there are pre-product or device steps to be performed. If “yes,” then processing proceeds to operation 725 where pre-product or device steps may be generated for Day X, Day Portion Y of the patient's schedule. If “no” at operation 720, processing proceeds to operation 730.

At operation 730, steps may be generated for the patient to apply a product or use a device. At operation 735, post-application steps may be generated for Day X, Day Portion Y. “Post-application steps,” as used herein, refers to steps to be performed by a patient after applying or using a product, such as using a treatment device followed by application of a particular product.

At operation 740, a determination is made regarding whether Day Portion Counter Y has reached a maximum value. For example, if there are two day portions for each day, the maximum value for Day Portion Counter Y may be a value of 2. If “no” at operation 740, Day Portion Counter Y may be incremented at operation 745 and processing may return to operation 715. In “yes” at operation 740, processing may advance to operation 750 where a determination may be made whether the value of Day Counter X has reached a maximum value. For example, if a schedule is determined for 28 days, the maximum value for Day Counter X may be 28.

If “no” at operation 750, Day Counter X may be incremented at operation 755, and processing may return to operation 710. If “yes” at operation 750, processing may proceed to operation 760, at which point the calendar for the patient is updated with the information determined in process 700 and processing may end.

In some embodiments, after a schedule has been generated for a patient, a skin care provider, such as a doctor or aesthetician, may view a calendar including the schedule and may edit one or more of the steps generated on any day and/or day portion. For example, a dermatologist may add in a prescribed medication and indicate that it should be applied once per day, in the morning, before applying sunscreen. Similarly, an aesthetician may alter one or more steps of a routine or product selection for a particular day and/or day portion. A skin care provider may access a patient's calendar via a skin care provider user interface to modify the patient's schedule or routine and/or product selection at any time in accordance with an embodiment. If the patient's routine and/or product selection is modified, the patient's calendar may be updated and the app or portal by which the patient accesses their schedule will show the updated information. This may involve deleting or invalidating any caches used by the system that store information for the patient.

Skin care providers may not be restricted to adding products and treatment devices for a patient. For example, skin care producers may have an ability to add arbitrary instructions of various types, such as to take medications, meditate, exercise, or perform other actions, along with instructions associated with those steps. For some of these, such as meditation, modules may dynamically construct appropriate text, generative audio or generative video. In some instances, text provided directly by a provider may be used.

To generate instructions, action types relating to an instruction may be determined and a related class may be called to determine how to find the instructions to inject into the patient's schedule or routine. For treatment devices, there may be a hierarchical-structure, such as JSON-based, for each device, which stores modalities and stores properties in the modalities for the device, and then stores instructions for each modality. A database may be accessed to find instructions in some instances. For example, for a particular treatment device, there may be one modality for using the treatment device by a patient who has acne, whereas there may be a different modality for using the treatment device by a patient who does not have acne. Depending on the modality, there may be different steps needed to use the associated treatment device.

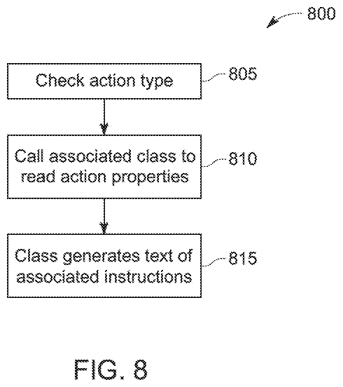

FIG. 8 illustrates a process 800 for determining instructions for a particular action to be performed according to an embodiment. Embodiments in accordance with claimed subject matter may include all of, less than, or more than blocks 805 through 815. Also, the order of blocks 805 through 815 is merely an example order. For example, a method in accordance with embodiment 800 may be performed by a computing device having one or more processors.

In one embodiment, process 800 may be implemented to automatically determine instructions associated with steps of the process discussed above with respect to process 700 of FIGS. A and B. For example, a module may be utilized to dynamically construct appropriate text, such as for a step in which a patient is to ingest, apply, or otherwise take a certain medication. In accordance with an implementation, an action type for a particular step may be determined and a related class may be called to determine how to find the instructions to inject for the step.

At operation 805, an action type may be checked for a step. For example, as discussed above, the automated determination of instructions may be limited to certain types of actions, such as taking or applying certain medicines. At operation 810, a class associated with the action type may be called to read or otherwise acquire action properties related to or associated with the action at issue. At operation 815, the class may generate text of the associated instructions. For example, the class may acquire the instructions relating to a particular medicine and may add those instructions to the patient's calendar or steps of a prescribed routine to perform.

In accordance with an embodiment, a patient's compliance to a prescribed schedule may be tracked or otherwise monitored. Such compliance tracking may ensure that the patient is keeping up with a prescribed schedule and may additionally enable a care provider to monitor how well the patient is following a prescribed routine. In some implementations, a user may be rewarded based on the patient's use of skin care or other products and/or the performance of actions relating to regular skin or hair care maintenance.

After a schedule of which actions, products, and treatment devices to use for a given part of the day, the schedule may be presented in a UI to the patient and the patient may mark off what they have done each day. The patient may be provided with reward points and may be reminded of what actions the patient has performed. This information may also be provided to care providers, so that the care providers may see whether the patient is following the desired routine or schedule. This aspect may be particularly important for a dermatologist who has prescribed prescription medicines, since the inputs from the patient may show the dermatologist the compliance of the patient. This feature may also feed a portion of a different algorithm which enables correlation of results in subsequent scans to user actions. That is, if the patient's moisture level gets better and it is known that the patient has been applying moisturizer regularly, it may be inferred that the patient's application of moisturizer was helpful, whereas if the patient's moisture level does not improve despite regularly applying moisturizer, it may be inferred that the patient needs to use a different moisturizer.

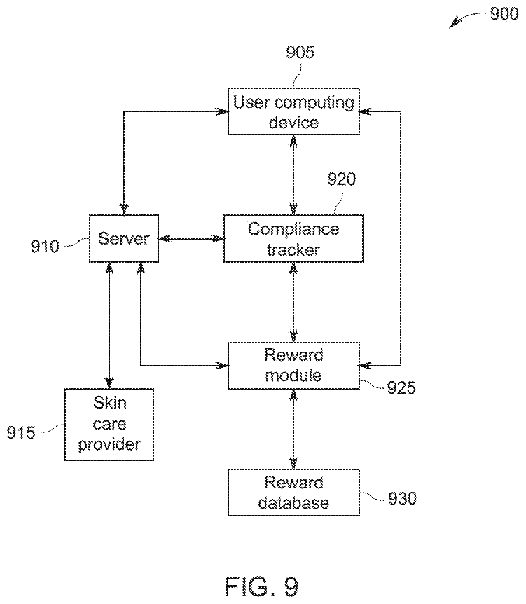

FIG. 9 illustrates a system 900 for monitoring a patient's compliance with a skin care routine and for providing rewards to the patient according to an embodiment. System 900 may be utilized to track the patient's performance of steps of a prescribed skin care routine or schedule and may provide rewards to the patient, such as provisioning loyalty points, a crypto coin, or some other form of reward to the patient. A reward may be utilized by the patient to purchase products or services at a discounted price, or at no charge in some implementations. In other implementations, a reward may be provided so that the patient can see how he or she has been following the schedule relative to how faithfully other patients have been following their prescribed schedules. A reward may be provided as an incentive to the patient to encourage the patient to follow the routine as well as an incentive to regularly report which actions the patient has performed regarding the routine so that a skin care provider may monitor the patient's progress.

In system 900, a patient may utilize a user computing device 905 to communicate the patient's performance of one or more actions relating to a daily schedule of a skin care routine. For example, the user computing device 905 may comprise a mobile device, such as a smart phone, on which a skin care application may be installed which may prompt the patient to perform certain actions and/or may request a user input when the user has performed certain actions, such as applying a moisturizer, for example. User computing device 905 may comprise a laptop or desktop computer in accordance with some embodiments, such as where the laptop or desktop computer runs an application program similar to that of a mobile device and which prompts the patient to perform certain actions and/or requests user inputs from the patient when certain actions have been performed. Such an application program may be associated with or may comprise a user portal through which the patient may communicate with a skin care provider, for example.

User inputs provided by the patient may be sent to or otherwise provided to a server 910. Server 910 may be accessible by a skin care provider 915 and or a business-to-business portal so that the skin care provider 915 or other entities may keep track of the patient's performance of a skin care routine. A compliance tracker 920 may be in communication with user computing device 905 and server 910. Compliance tracker 920 may maintain records relating to a patient's performance of actions relating to a prescribed skin care routine and may determine how closely the patient is following a prescribed routine or schedule. For example, compliance tracker 920 may calculate a score for a patient based on how closely the patient is following a prescribed routine. A calculated score may be provided to a reward module 925. Reward module 925 may be in communication with a reward database 930. Various rewards may be stored in reward database 930, such as vouchers or coupons for services or products, badges which a patient may display on a profile page for the patient in a portal, and/or any other form of suitable reward to motivate the patient. Reward module 925 may obtain a reward from reward database 930 based on the calculated score for the patient and may provide the reward to user computing device 905, for example.

Patients with the highest scores may comprise the patients who have been performing the most skin care routines. Such patients may be highly valuable to certain businesses, such as spa treatment facilities or cosmetic retailers. Accordingly, a spa treatment facility or cosmetic retailer may choose to offer rewards to patients with the highest scores because those patients may be highly valuable future customers, for example.

In accordance with an embodiment, a user's schedule may be recomputed periodically, such as every N days. The patient may continually upload scans and selfie photos on a regular basis and based on changes detected within the scans and selfie images, it may be determined that the patient's skin condition is improving such that the patient may need to use certain products less often, such as moisturizers, for example, or that the patient's condition is not improving and the routine and products should be changed.

In accordance with an embodiment, a personalized audio track and/or a personalized video track may be generated for a patient to provide instructions and/or motivation to the patient to perform the desired skin care routine. A personalized “track,” as used herein, refers to a file containing audio and/or video. This audio track and/or video track can enforce the steps and timings needed for proper skin care, which are often ignored or not known by patients. In addition, the audio track and/or video track may help create a consistent ritual for the patient that the patient can look forward to, helping to keep the patient motivated and engaged. One way to provide proper instructions and additional motivation to keep the patient interested in continuing to follow a prescribed skin care routine or schedule is by generating a personalized audio track and/or personalized video track and providing a link, such as a Uniform Resource Locator (URL), to the personalized track accessible via a skin care application running on the patient's mobile device, computer, television, or on some other electronic device capable of presenting audio, such as Alexa™.

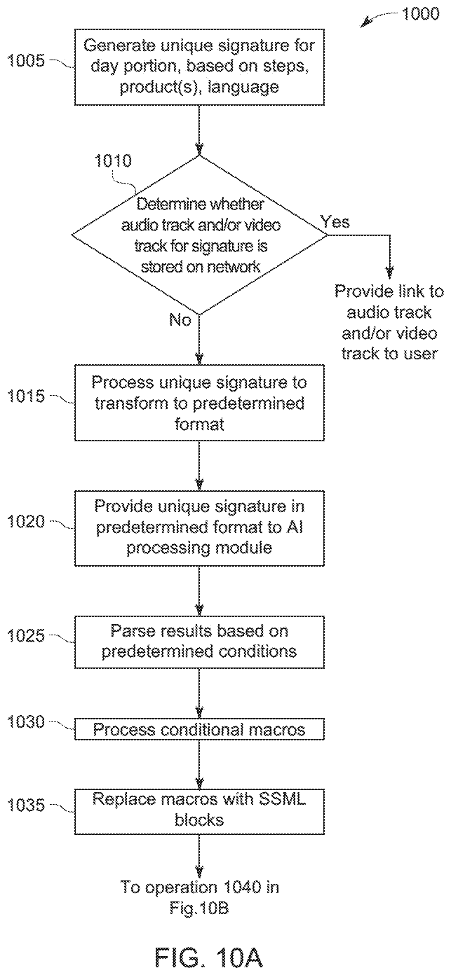

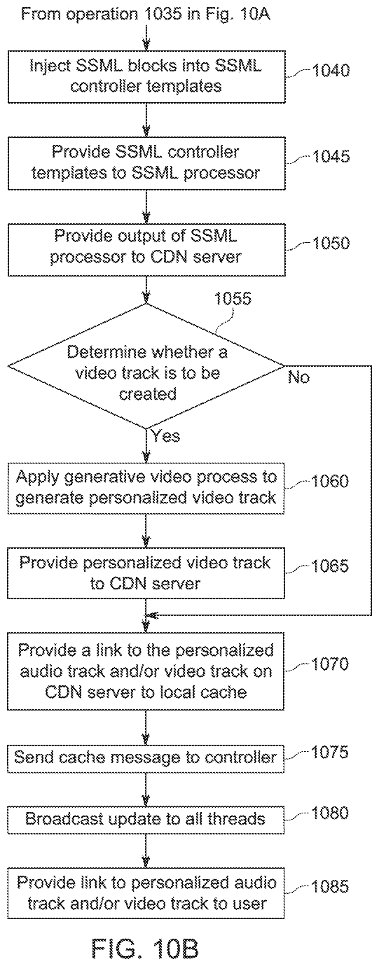

FIGS. 10A and 10B illustrate a process 1000 for providing a link to a personalized audio track and/or video track to a skin care patient according to an embodiment. At operation 1005, a unique signature may be generated for a day portion of a patient's scheduled routine, where the unique signature is based on the various steps that the patient is to perform during that day portion, the product(s) to be applied, and the language of the audio to be provided. In accordance with an embodiment and as previously discussed above, a schedule or routine for a patient may comprise a multi-day schedule where each day is segmented into multiple day portions, such as a morning day portion, an evening day portion, and/or an afternoon day portion. For example, a personalized audio track may be generated in a plurality of different languages, such as American English, British English, French, Spanish, German, Mandarin Chinese, or Portuguese, to name just a few languages among many. The personalized audio track may also be generated in the manner of a character on a tv show or movie, for example, such as in the style of a character from the Sopranos™ or Star Wars™.

At operation 1010, a determination is made as to whether the audio track and/or video track for the signature has previously been generated and is already stored on a network, such as in either a local cache or within a Content Delivery Network (CDN) storage device which is capable of sending a link to the audio track to the local cache storage device. If “yes” at operation 1010, a link to the audio for the signature may be provided to the patient. For example, a URL may be presented to the patient via an application program or via a user interface, which the user may select in order to cause the personalized audio track and/or video track to be played to the patient. If “no,” on the other hand at operation 1010, the personalized audio track and/or video track may be created for the patient.

At operation 1015, the combination of products, steps, languages, style and other factors may be transformed into text via a generator template. A generator template may comprise data or other information organized in a particular way so that it may generate full text instructions or it may be designed to provide instructions designed for further processing by a generative AI module. In both cases, the template may contain substitution macros that are used to incorporate the specific context (products, languages, products, user name, etc.) into the generated text. For example, as part of the transformation, substitution macros may be processed to inject the patient's name, the time of day, product names, product application instructions, or other information into the template so as to make the output customized for the particular context. An additional part of the transformation may include iterating through all treatment devices associated with the day part at issue and inject instructions into the generator template, such as instructions indicating how each treatment device is to be utilized.

At operation 1020, the output of the template may be additionally transformed to use generative AI, such as ChatGPT™ or a different LLM, to construct detailed textual instructions. For example, the AI processing module may be provided with a request to generate skin care routine instructions in a meditative mood with a speech pattern similar to Marilyn Monroe's phrasing and vocabulary, in French, and which includes the specific named products and for a particular skin care device, and in which certain known text instructions are to be spoken. The AI processing module may serve to generate instructions in a style or manner which makes listening to the personalized audio track more entertaining, for example. The AI processing module may also generate instructions in accordance with various themes or modes, such as a holiday mode. The AI processing module may therefore serve to keep the text instructions and the corresponding generated audio track fresh instead of playing the exact same audio track every day.

The AI processing may need to undergo additional transformations before it may be used for generating an audio track. For example, the output might not be in a conversational format. For example, the generated audio instructions may recite “bullet one, apply facial cleanser in a circular motion for ten seconds, semicolon, bullet two . . . ” The output from the AI processing module may be processed to clean up the generated audio and remove unnecessary preambles or other undesirable information and to turn the text into a style more appropriate for spoken instructions.

At operation 1025, results from the AI processing module may be parsed based on certain predetermined conditions. For example, if there are timing cues within the generated text, delay instructions may be introduced based on the timing cues. In accordance with an implementation, if the generated audio recites “put moisture on for 30 seconds. Now put on your skin cream. Now put on sunscreen,” certain timing breaks may be introduced so that the instruction to apply skin cream is delayed for at least 30 seconds after the instruction to apply moisturizer for 30 seconds has been presented, so that the patient has a sufficient amount of time to apply the moisturizer for 30 seconds before the next instruction to apply skin cream is presented to the patient. Similarly, the instruction to apply sunscreen may also be delayed, such as by several minutes, in order to give the patient a sufficient amount of time to finish applying the skin cream prior to the instruction to apply sunscreen is presented to the patient. Delays may be added between paragraphs for appropriate spoken pauses. During this step the text is scanned, in whatever language it was generated in, for appropriate signals for timing information. These could be explicit as in the previous examples, or they could be more generic such as “let sit for a few minutes.” There may be other transformations in this step that enable the text to be structured in a format that will work with text-to-speech audio generation. This incorporation of timing information enables the text instructions to be transformed to a style appropriate for audio conversion so that the user can listen to the instructions and follow along while performing their skin care routine.

Sound generation and AI may be expensive. Accordingly, in order to keep costs down, the generated audio tracks and/or video tracks may be stored in a cache so that they do not need to be constantly created separately for each individual patient. For every audio track and/or video track generated, there may be a unique signature value and a check may be performed to determine whether the signature has previously been created and is currently stored in the local cache.

At operation 1030, processing may be performed for conditional macros. For example, there may be macros which indicate that if a certain hardware device is used, that certain operations are to be performed. For example, audio instructions such as “I see that you are using your facial wand device today” may be presented to the patient. The audio instructions may be smart based on that day's events. The audio instructions may state the exact products which the patient is known to be using that day and may be generated for certain languages. For example, if the patient is a female, a female voice in Spanish language may be used if desired.

At operation 1035, the text and timing information may be replaced with a format designed for audio synthesis. One such format is the Speech Synthesis Markup Language (SSML). SSML is a markup language which provides a standardized way to mark up text for the generation of synthetic speech. SSML or similar markup languages includes syntax for controlling timing, voice, emphasis, background audio and more and the transformation from the intermediate text generated in operation 1030 into the markup language prepares the system for audio generation.

In one example, the audio generation process may start with a general instruction or data, such as “use Alastin Blue Moisturizer™” From this, a template may be employed that generates explicit text, such as “Apply [product name] for 30 seconds” or that generates something designed for generative AI such as “create instructions for [product list] in [language].” After generating the text, timing or other signals may be incorporated into the generated text. For example, incorporating timing or other signals would result in generated text such as “Apply Alastin Blue Moisturizer™ for 30 seconds<<delay 30>>” where <<delay 30>> is a macro which has been built to incorporate the timing information. After incorporating the timing or other signals, the text may be turned into SSML. Turning or converting text into SSML is akin to turning a Word™ document into HTML, such as where general text may be converted to an SSML syntax. After converting to SSML, additional preamble SSML information may be incorporated that includes audio generation guidelines (such as, e.g., voices, background sound, and so forth). After a full SSML document has been generated, the SSML document may be provided to an audio generation service which converts the SSML document into an audio track or file.

Referring back to embodiment 1000, at operation 1040, the generated SSML content fragment from operation 1035 may be incorporated into a broader SSML template that incorporates necessary SSML preamble tags, additional tags for voice selection, background sound track selection, volume or speed controls, and other overall stylistic information for the audio generation. This provides a full SSML document (instead of a fragment) that is built from the text generated in the previous steps and is then ready to be sent to an SSML processing service for audio generation. At operation 1045, the SSML document may be provided to an SSML processor which may render an audio file from the textual information.

At operation 1050, the output of the SSML processor may be provided to a CDN server. For example, the audio file may be stored on the CDN server or may be stored in a storage device, such as a database, accessible by the CDN server.

The audio file that is generated may also be used for construction of a video file. First, the audio file may be generated and propagated to the CDN server before the video file may be created in accordance with an environment. At operation 1055, a determination may be made as to whether a video file is to be created. If “No,” processing proceeds to operation 1070. If, “Yes,” a generative video process may be applied to generate a personalized video track at operation 1060. For example, an API may be used, such as the HeyGen—AI Video Generator™, the D-ID™—AI video platform, or a lower level service such as Amazon SageMaker™. If a customer has previously uploaded head shots of a treatment provider (e.g., such as an aesthetician or dermatologist) as part of a portal configuration, those images may be used as seed images for generating video content. If multiple images have been provided, one of the images may be selected at random so that the imagery remains fresh for the patient. On the other hand, if no images have been provided, a preconfigured avatar may be employed with a gender selected to match the patient's preferences. The audio track generated at operations 1015-1050 may be used as an audio input for the video track. A generated video track may have a file format such as Moving Picture Experts Group (MPEG)-4 Part 14 (“MP4”) or any other suitable video file format.

At operation 1065, the personalized video track may be provided to a CDN server. At operation 1070, a link to the audio track and/or video track on the CDN server may be provided to a local cache. At operation 1075, a new cache message may be sent to a controller to inform the controller of the presence of a newly cached link. At operation 1080, the controller may broadcast an update to all processing threads to inform the threads of the link to the new audio track and/or video track, for example. At operation 1085, a link to the audio track and/or video track may be presented to a patient, such as via a skin care routine application and/or other a skin care routine user interface or portal.

In addition to generating video as part of the scheduled instructions, video may be particularly useful for providing new instructions to a patient. For example, if as part of the customization of steps, a skin care provider adds new instructions for a customer, such as for a medication or a new skin care product or exercise routine, a generative video specific to that instruction may be generated. A special instruction template may be incorporated, such as using the same technology steps described above with respect to embodiment 1000 of FIGS. 10A and 10B, with a template containing instructions such as “[skin care provider name] has added a special new [diet/treatment/exercise] step for your regimen,” followed by the specific treatment steps. This process may generate an audio track or file specific to the one new step along with the video, and may be promoted into special areas of a portal, such as an area for news and other announcements, so that the new instructions will receive extra attention in addition to their presence in the normal audio track. In this specific instance, using the new video track may provide a strong visual signal of the importance of the step, and may provide an experience familiar to patients who view videos on Instagram™ and other sites that often contain beauty related instructions.

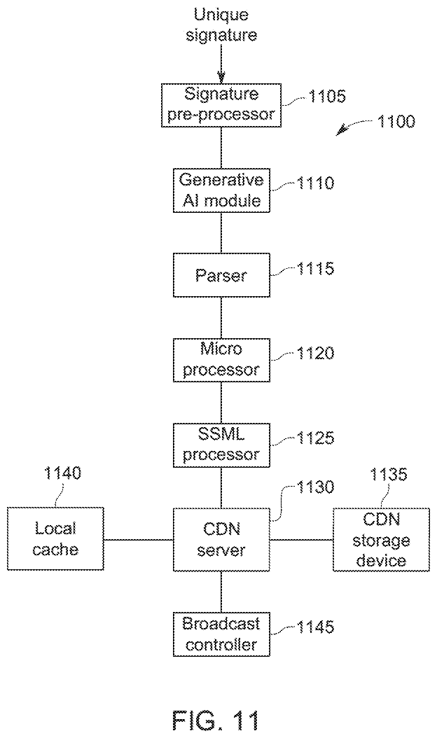

FIG. 11 illustrates a system 1100 for implementing a process for providing a link to a personalized audio track to a skin care patient according to an embodiment. For example, system 1100 may implement process 1000 described above with respect to FIGS. 10A and 10B. A signature pre-processor 1105 may receive a unique signature for audio to be generated, such as is performed in operation 1015 of process 1000. A generative AI module 1110 may receive an output from signature pre-processor 1105 and may generate and output a text file, such as is performed in operation 1020 of process 1000. A parser 1115 may take the output from generative AI module 1110 and may parse the results based on predetermined conditions, such as is performed at operation 1025 of process 1000. Macro processor 1120 may process additional macros, replace macros with SSML blocks, and inject the SSML blocks into SSML controller templates, such as is performed in operations 1030-1045 of process 1000. SSML processor 1125 may generate an audio file based on the SSML controller templates, such as is performed at operation 1045 of process 1000. The output of SSML processor 1125 may be provided to a CDN server 1130 which may store the audio file on itself or in a CDN storage device 1135, such as a database. CDN server 1130 may generate a link to the audio file and may provide the link to local cache 1140 for storage. A broadcast controller 1145 may broadcast an update to all threads, such as of a multi-thread processor.

Some portions of the detailed description are presented herein in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular functions pursuant to instructions from program software. Algorithmic descriptions or symbolic representations are examples of techniques used by those of ordinary skill in the signal processing or related arts to convey the substance of their work to others skilled in the art. An algorithm is here, and generally, considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated.

It has proven convenient at times, principally for reasons of common usage, to refer to such signals as bits, data, values, elements, symbols, characters, terms, numbers, numerals or the like. It should be understood, however, that all of these or similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

It should be understood that for ease of description, a network device (also referred to as a networking device) may be embodied and/or described in terms of a computing device. However, it should further be understood that this description should in no way be construed that claimed subject matter is limited to one embodiment, such as a computing device and/or a network device, and, instead, may be embodied as a variety of devices or combinations thereof, including, for example, one or more illustrative examples.

The terms, “and”, “or”, “and/or” and/or similar terms, as used herein, include a variety of meanings that also are expected to depend at least in part upon the particular context in which such terms are used. Typically, “or” if used to associate a list, such as A, B or C, is intended to mean A, B, and C, here used in the inclusive sense, as well as A, B or C, here used in the exclusive sense. In addition, the term “one or more” and/or similar terms is used to describe any feature, structure, and/or characteristic in the singular and/or is also used to describe a plurality and/or some other combination of features, structures and/or characteristics. Likewise, the term “based on” and/or similar terms are understood as not necessarily intending to convey an exclusive set of factors, but to allow for existence of additional factors not necessarily expressly described. Of course, for all of the foregoing, particular context of description and/or usage provides helpful guidance regarding inferences to be drawn. It should be noted that the following description merely provides one or more illustrative examples and claimed subject matter is not limited to these one or more illustrative examples; however, again, particular context of description and/or usage provides helpful guidance regarding inferences to be drawn.

A network may also include now known, and/or to be later developed arrangements, derivatives, and/or improvements, including, for example, past, present and/or future mass storage, such as network attached storage (NAS), a storage area network (SAN), and/or other forms of computing and/or device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as computing and/or communication protocols (e.g., network protocols), may interoperate within a larger network. In this context, the term sub-network and/or similar terms, if used, for example, with respect to a network, refers to the network and/or a part thereof. Sub-networks may also comprise links, such as physical links, connecting and/or coupling nodes, such as to be capable to transmit signal packets and/or frames between devices of particular nodes, including wired links, wireless links, or combinations thereof. Various types of devices, such as network devices and/or computing devices, may be made available so that device interoperability is enabled and/or, in at least some instances, may be transparent to the devices. In this context, the term transparent refers to devices, such as network devices and/or computing devices, communicating via a network in which the devices are able to communicate via intermediate devices of a node, but without the communicating devices necessarily specifying one or more intermediate devices of one or more nodes and/or may include communicating as if intermediate devices of intermediate nodes are not necessarily involved in communication transmissions. For example, a router may provide a link and/or connection between otherwise separate and/or independent LANs. In this context, a private network refers to a particular, limited set of network devices able to communicate with other network devices in the particular, limited set, such as via signal packet and/or frame transmissions, for example, without a need for re-routing and/or redirecting transmissions. A private network may comprise a stand-alone network; however, a private network may also comprise a subset of a larger network, such as, for example, without limitation, all or a portion of the Internet. Thus, for example, a private network “in the cloud” may refer to a private network that comprises a subset of the Internet, for example. Although signal packet and/or frame transmissions may employ intermediate devices of intermediate nodes to exchange signal packet and/or frame transmissions, those intermediate devices may not necessarily be included in the private network by not being a source or destination for one or more signal packet and/or frame transmissions, for example. It is understood in this context that a private network may provide outgoing network communications to devices not in the private network, but devices outside the private network may not necessarily be able to direct inbound network communications to devices included in the private network.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all implementations falling within the scope of the appended claims, and equivalents thereof.

What is claimed is:

1. A method, comprising:
receiving selfie images of a patient;
receiving additional images of one or more portions of the patient's body from a skin measurement/monitoring device;
processing the selfie images and the additional images to identify one or more skin care conditions for the patient;
determining group of skin care products to recommend to the patient based on the identified one or more skin care conditions for the patient;
generating a multi-day, multi-day portion schedule for a skin care routine for the patient, the skin care routine comprising one or more products from the group of skin care products and one or more additional actions for the patient to perform;
generating instructions for patient, the instructions indicating how to use the one or more products and how to perform the one or more additional actions in a particular order;
transforming the instructions into a personalized track comprising at least one of audio or video; and
providing a link to the personalized track to the patient.

2. The method of claim 1, further comprising utilizing an Artificial Intelligence module to generate an audio output comprising one or more of the instructions.

3. The method of claim 2, wherein the audio output is in a particular language selected for the patient and includes background music corresponding to a particular mood.

4. The method of claim 2, wherein the audio output is generated in accordance with a selected audio theme.

5. The method of claim 2, further comprising processing the audio output to insert one or more time breaks for audio playback.

6. The method of claim 1, wherein the determining of the group of skin care products to recommend comprising filtering out one or more skin care products from consideration based on one or more of product categories, product tags, or product ingredients.

7. The method of claim 1, wherein the determining of the group of skin care products to recommend comprising determining particular skin care products owned by the patient and filtering out working rival products from consideration.

8. The method of claim 1, further comprising instructing the patient to capture new selfie images or more new additional images of the one or more portions of the patient's body from a skin monitoring device.

9. The method of claim 1, further comprising periodically recalculating the multi-day, multi-day portion schedule for a skin care routine for the patient based, in part, on the new selfie images or the new additional images of the one or more portions of the patient's body.

10. The method of claim 1, enabling a skin care provider to access the selfie images and the additional images for the patient via a skin care portal.

11. The method of claim 1, further comprising providing one or more rewards to the patient in exchange for the patient providing user inputs indicating that the patient is using the one or more products or performing one or more additional actions of the skin care routine for the patient.

12. A system comprising:
at least one programmable processor; and
a non-transitory machine-readable medium storing instructions that, when executed by the at least one programmable processor, cause the at least one programmable processor to perform operations comprising:
processing selfie images of a patient and the additional images of one or more portions of the patient's body from a skin measurement/monitoring device to identify one or more skin care conditions for the patient;
determining group of skin care products to recommend to the patient based on the identified one or more skin care conditions for the patient;
generating a multi-day, multi-day portion schedule for a skin care routine for the patient, the skin care routine comprising one or more products from the group of skin care products and one or more additional actions for the patient to perform;
generating instructions for patient, the instructions indicating how to use the one or more products and how to perform the one or more additional actions in a particular order;
transforming the instructions into a personalized track comprising at least one of audio or video; and
providing a link to the personalized track to the patient.

13. The system of claim 12, wherein the instructions are further executable by the at least one programmable processor to perform at least one additional operation comprising utilizing an Artificial Intelligence module to generate an audio output comprising one or more of the instructions.

14. The system of claim 12, wherein the instructions are further executable by the at least one programmable processor to perform at least one additional operation comprising processing the audio output to insert one or more time breaks for audio playback.

15. The system of claim 12, wherein the instructions are further executable by the at least one programmable processor to perform at least one additional operation comprising filtering out one or more skin care products from consideration based on one or more of product categories, product tags, or product ingredients.

16. The system of claim 12, wherein the instructions are further executable by the at least one programmable processor to perform at least one additional operation comprising determining particular skin care products owned by the patient and filtering out working rival products from consideration.

17. The system of claim 12, wherein the instructions are further executable by the at least one programmable processor to perform at least one additional operation comprising instructing the patient to capture new selfie images or more new additional images of the one or more portions of the patient's body from a skin monitoring device.

18. The system of claim 12, wherein the instructions are further executable by the at least one programmable processor to perform at least one additional operation comprising periodically recalculating the multi-day, multi-day portion schedule for a skin care routine for the patient based, in part, on the new selfie images or the new additional images of the one or more portions of the patient's body.

19. The system of claim 12, wherein the instructions are further executable by the at least one programmable processor to perform at least one additional operation comprising enabling a skin care provider to access the selfie images and the additional images for the patient via a skin care portal.

20. The system of claim 12, wherein the instructions are further executable by the at least one programmable processor to perform at least one additional operation comprising providing one or more rewards to the patient in exchange for the patient providing user inputs indicating that the patient is using the one or more products or performing one or more additional actions of the skin care routine for the patient.

21. An article, comprising:
a non-transitory storage medium comprising machine-readable instructions executable by a processor to perform:
processing selfie images of a patient and the additional images of one or more portions of the patient's body from a skin measurement/monitoring device to identify one or more skin care conditions for the patient;
determining group of skin care products to recommend to the patient based on the identified one or more skin care conditions for the patient;
generating a multi-day, multi-day portion schedule for a skin care routine for the patient, the skin care routine comprising one or more products from the group of skin care products and one or more additional actions for the patient to perform;
generating instructions for patient, the instructions indicating how to use the one or more products and how to perform the one or more additional actions in a particular order;
transforming the instructions into a personalized track comprising at least one of audio or video; and
providing a link to the personalized track to the patient.
